(19) United States
(12) Patent Application Publication
(10) Pub. No.: US 2022/0079934 A1
(43) Pub. Date: Mar. 17, 2022

(54) CRENOLANIB FOR TREATING PAIN

(71) Applicant: Arog Pharmaceuticals, Inc., Plano, TX (US)

(72) Inventor: Vinay K. Jain, Dallas, TX (US)

(21) Appl. No.: 17/320,409

(22) Filed: May 14, 2021

Related U.S. Application Data
(60) Provisional application No. 63/079,602, filed on Sep. 17, 2020.

(51) Int. Cl. A61K 31/4709

(52) U.S. Cl. CPC A61K 31/4709

(57) ABSTRACT

The present invention includes methods for reducing or eliminating pain a subject comprising administering to the subject a therapeutically effective amount of crenolanib or a pharmaceutically acceptable salt thereof

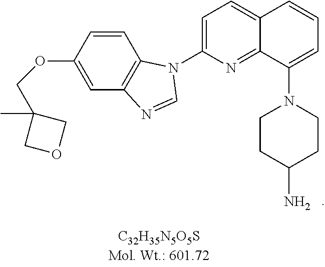 [SMILES: CC1(COc2ccc3c(c2)ncn3-c2ccc3cccc(N4CCC(N)CC4)c3n2)COC1]

### CROSS-REFERENCE TO RELATED APPLICATIONS

This application claims priority to U.S. Provisional Application Ser. No. 63/079,602, filed Sep. 17, 2020, the entire contents of which are incorporated herein by reference.

### TECHNICAL FIELD OF THE INVENTION

The present invention includes a method of treating pain in a subject using a compound of crenolanib or a pharmaceutically acceptable salt thereof.

### STATEMENT OF FEDERALLY FUNDED RESEARCH

Not applicable.

### INCORPORATION-BY-REFERENCE OF MATERIALS FILED ON COMPACT DISK

Not applicable.

### BACKGROUND OF THE INVENTION

Without limiting the scope of the invention, its background is described in connection with its ability to inhibit the tropomyosin receptor (TRK) kinases in the treatment of pain or pain related disorders.

The TRK kinases, TRKA, TRKB, and TRKC are involve in a number of normal physiological processes including cell growth and proliferation, neurite outgrowth, pain, and sensory modulation. Each kinase is encoded on a separate gene, NTRK1, NTRK2, and NTRK3, respectively (Amatu, Sartore-Bianchi et al. 2019). In normal signalling, TRK kinases are activated by neurotrophins, including: nerve growth factor (NGF) which activates TRKA; brain derived neurotrophic factor (BDNF) which activates TRKB; neurotrophin 3 (NT-3) which activates TRKA, TRKB, and TRKC; and neurotrophin 4 (NT-4) which activates TRKB (Amatu, Sartore-Bianchi et al. 2019). When a ligand binds the partner TRK receptor kinase, it induces receptor dimerization, autophosphorylation, and downstream signalling, a common activation sequence in receptor tyrosine kinases.

Signaling through NGF and TRKA has been associated with pain sensing in both animal models and human patients (Hirose, Kuroda et al. 2016). Loss of function mutations in TRKA cause congenital insensitivity to pain with anhidrosis, a condition in which patients lack nociceptive and sympathetic nerves and do not feel pain. These patients also are unable to sweat, which leads to repetitive injuries and overheating upon exercise (Indo, Tsuruta et al. 1996). A variant of this disorder in which patients have an insensitivity to pain without anhidrosis, hereditary sensory and autonomic neuropathy type 5, is associated with mutations in NGF (Einarsdottir, Carlsson et al. 2004).

There are two types of physical pain: nociceptive and neuropathic pain. Nociceptive pain is the result of damage to tissues such as muscle, skeletal, or skin and activates nociceptors. Neuropathic pain is the result of disease or lesions on the nervous system (Loeser and Treede 2008). NGF activation of TRKA is involved in both types of pain. NGF is secreted by inflammatory cells after tissue injury, and local up-regulation of NGF has been found in human patients presenting with fibromyalgia, migraines, arthritis, and other chronic pain disorders (Aloe, Tuveri et al. 1992, Sarchielli, Mancini et al. 2007). Moreover, intravenous or subcutaneous administration of NGF induces hyperalgesia in humans, with muscle pain and hyperalgesia of the skin lasting for up to several weeks (Petty, Cornblath et al. 1994). The involvement of TRKA and NGF signaling in has made inhibition of this pathway an attractive target in the treatment of acute pain, as well as chronic pain disorders.

Current pharmaceutical treatments for pain carry significant risks for chronic use. Cyclooxygenase-2 inhibitors such as acetaminophen carry cardiovascular and liver damage risk with long-term sustained use. Chronic use of nonsteroidal anti-inflammatory drugs (NSAIDs) such as ibuprofen is associated gastrointestinal and renal side effects (Mantyh, Koltzenburg et al. 2011). While opioids can significantly reduce pain, chronic use of these compounds carries a significant risk for substance abuse, addiction, and accidental overdose as well as side effects such as loss of drug effectiveness and constipation (Mantyh, Koltzenburg et al. 2011). These issues further motivate the search for more effective, safe, agents in the treatment of chronic pain.

In an animal model of inflammatory arthritis, treatment with a TRKA specific inhibitor reduced pain behavior, such as weight bearing asymmetry, punctate allodynia, or paw withdrawal. Treatment also slowed joint swelling, synovial inflammation, and joint damage (Ashraf, Bouhana et al. 2016). Initial human studies of the monoclonal antibody tanezumab, which blocks binding of NGF to the TRKA receptor, was initially studied in osteoarthritis and had positive results on chronic pain. However, the United States Food and Drug Administration placed a clinical hold on further development of this agent in 2012 when there were reports of patients' development of osteonecrosis (Hochberg, Tive et al. 2016). Adjudication of these events by a blinded committee of orthopedic surgeons, rheumatologists, and an orthopedic pathologist found that tanezumab treatment was not associated with osteonecrosis, but rather that the events reported as such were progressing osteoarthritis. Further, in patients reporting rapid progression of osteoarthritis, coadministration with NSAIDs and pre-existing subchondral insufficiency fractures were found to significantly contribute to the development of this adverse effect (Hochberg, Tive et al. 2016).

The discovery that patients treated with tanezumab did not in fact develop osteonecrosis led the FDA to lift the clinical hold, and clinical trials moved forward with a comprehensive risk minimization strategy, prohibiting coadministration of NSAIDs, using a lowered dose of tanezumab, excluding patients with risk factors for rapidly progressive osteoarthritis, and excluding patients who would not be eligible for total joint replacement. Patients treated with these safety measures in place reported significant decrease in pain compared to patients receiving placebo, however, safety events including arthralgia, join swelling, paresthesia, and back pain were reported in a significant number of patients (Schnitzer, Easton et al. 2019). A total of 22 patients (9.4%) treated with tanezumab required knee or joint replacement, indicating that this agent is not ideal for all patients (Schnitzer, Easton et al. 2019). The lower dose used in more recent trials is believed to offset a problem seen at higher doses, namely, that patients were overexerting their damaged joints due to the lack of pain. While tanezumab relieves the pain associated with osteoarthritis, it does not heal the damage, and patient overexertion led to further joint damage and the need for joint replacement. This is analogous to patients with congenital insensitivity to pain getting injured. In some cases, pain is a sign that a certain activity should not be performed. The lack of such a signal may lead to injury. A pharmaceutical agent which relieves pain associated with osteoarthritis or other disorders, but not to the extent that normal pain signals are non-existent or easily ignored, maybe the ideal solution for the unmet need in this population.

Another anti-NGF antibody, fasinumab, has also been studied in clinical trials of osteoarthritis pain relief. As with tanezumab, a significant number of patients experienced arthralgia or rapidly progressive osteoarthritis (Dakin, DiMartino et al. 2019). Moreover, as monoclonal antibodies, both tanezumab and fasinumab carry the risk of side effects associated with this drug class, including anaphylaxis, serum sickness, and the generation of antibodies that may lead to autoimmune disease (Hansel, Kropshofer et al. 2010). In addition, monoclonal antibodies, including tanezumab and fasinumab, are administered by injection every 4-8 weeks, requiring patients to coordinate numerous appointments with their physician. Pharmaceutical agents that can be given orally increase patient compliance and decrease the substantial burden placed on patients when other options require a doctor visit for drug administration.

Given the significant side effects associated with monoclonal antibodies in general, and tanezumab and fasinumab in particular, as well as the burden and inconvenience of administration by injection, an orally bioavailable agent that inhibits the NGF-TRKA signalling axis and relieves pain associated with osteoarthritis, fibromyalgia, and other pain disorders, would be of benefit to patients.

### SUMMARY OF THE INVENTION

In one embodiment, the present invention includes a method for reducing or eliminating pain in a subject comprising administering to the subject a therapeutically effective amount of crenolanib or a pharmaceutically acceptable salt thereof wherein the crenolanib or salt thereof relieves the symptom of pain. In one aspect, the inhibition of TRKA kinase attenuates or ameliorates pain. In another aspect, the pain is selected from at least one of osteoarthritis pain, chronic pain, neuropathic pain, musculoskeletal pain, acute pain, inflammatory pain, pain associated with a proliferative disease or disorder, idiopathic pain, postsurgical pain, visceral pain, multiple sclerosis, pain associated with bone fracture, trigeminal neuralgia, rheumatoid arthritis, and fibromyalgia. In another aspect, the therapeutically effective amount or crenolanib or the pharmaceutically acceptable salt thereof is from about 50 to 800 mg per day, 100 to 450 mg per day, 200 to 400 mg per day, 300 to 500 mg per day, 350 to 500 mg per day, 400 to 600 mg per day, or 600 to 800 mg per day. In another aspect, the therapeutically effective amount of crenolanib or the pharmaceutically acceptable salt thereof is administered at least one of continuously, intermittently, systemically, or locally. In another aspect, the crenolanib or the pharmaceutically acceptable salt thereof is administered orally, intravenously, intraperitoneally, intramuscularly, subcutaneously, or topically. In another aspect, the crenolanib or the pharmaceutically acceptable salt thereof is administered as a single agent, or concomitantly or sequentially with another pharmaceutical agent. In another aspect, the crenolanib is a pharmaceutically acceptable salt of crenolanib. In another aspect, the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, crenolanib tosylate, crenolanib mesylate, crenolanib benzoate, or crenolanib succinate. In another aspect, the therapeutically effective amount of crenolanib or the pharmaceutically acceptable salt thereof is administered up to three times or more a day for as long as the subject is in need of treatment.

In one embodiment, the present invention includes a method for reducing or eliminating pain in a subject comprising identifying a subject in need of reducing or eliminations pain and administering to the subject a therapeutically effective amount of crenolanib or a pharmaceutically acceptable salt thereof wherein the crenolanib or salt thereof to reduce or eliminate pain. In one aspect, the inhibition of TRKA kinase attenuates or ameliorates pain. In another aspect, the pain is selected from at least one of osteoarthritis pain, chronic pain, neuropathic pain, musculoskeletal pain, acute pain, inflammatory pain, pain associated with a proliferative disease or disorder, idiopathic pain, postsurgical pain, visceral pain, multiple sclerosis, pain associated with bone fracture, trigeminal neuralgia, rheumatoid arthritis, and fibromyalgia. In another aspect, the therapeutically effective amount or crenolanib or the pharmaceutically acceptable salt thereof is from about 50 to 800 mg per day, 100 to 450 mg per day, 200 to 400 mg per day, 300 to 500 mg per day, 350 to 500 mg per day, 400 to 600 mg per day, or 600 to 800 mg per day. In another aspect, the therapeutically effective amount of crenolanib or the pharmaceutically acceptable salt thereof is administered at least one of continuously, intermittently, systemically, or locally. In another aspect, the crenolanib or the pharmaceutically acceptable salt thereof is administered orally, intravenously, intraperitoneally, intramuscularly, subcutaneously, or topically. In another aspect, the crenolanib or the pharmaceutically acceptable salt thereof is administered as a single agent, or concomitantly or sequentially with another pharmaceutical agent. In another aspect, the crenolanib is a pharmaceutically acceptable salt of crenolanib. In another aspect, the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, crenolanib tosylate, crenolanib mesylate, crenolanib benzoate, or crenolanib succinate. In another aspect, the therapeutically effective amount of crenolanib or the pharmaceutically acceptable salt thereof is administered up to three times or more a day for as long as the subject is in need of treatment.

### BRIEF DESCRIPTION OF THE DRAWINGS

For a more complete understanding of the features and advantages of the present invention, reference is now made to the detailed description of the invention along with the accompanying figures and in which:

FIG. 1 shows the dose-response curve for IC50 determination of the besylate salt of the present invention for wildtype TRKA. The activity of the besylate salt of crenolanib is plotted against the corresponding molar concentration in log 10 scale.

FIG. 2 shows the dose-response curve for IC50 determination of the besylate salt of the present invention for wildtype TRKB. The activity of the besylate salt of crenolanib is plotted against the corresponding molar concentration in log 10 scale.

FIG. 3 shows the dose-response curve for IC50 determination of the besylate salt of the present invention for wildtype TRKC. The activity of the besylate salt of crenolanib is plotted against the corresponding molar concentration in log 10 scale.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

The present invention is directed to the administration of crenolanib, or a pharmaceutically acceptable salt thereof, to subjects suffering from a pain disorder or pain associated with another disease or disorder in order to attenuate or ameliorate the pain in a subject.

Crenolanib is an orally bioavailable TKI. It is significantly more selective for PDGFR, FLT3, and TRK kinases than other kinases, including c-KIT, VEGFR2, TIE2, FGFR2, EGFR, ERBB2, and SRC (Lewis, Lewis et al. 2009, Aikawa, Togashi et al. 2020). As a type 1 TKI which directly interacts with ATP binding pocket, it binds to both the active and inactive conformations of the kinase. Importantly, crenolanib shows clinical activity against FLT3 and PDGFRA mutated proliferative disorders, and has a promising safety profile in both solid tumors and hematological malignancies (von Mehren, Tetzlaff et al. 2016, Goldberg 2020). The preclinical data presented herein confirm that crenolanib also effectively inhibits TRKA. As such, crenolanib is ideally suited for the treatment of pain associated with TRKA signalling.

The present invention comprises methods of attenuating or ameliorating pain in a subject through the inhibition of TRKA kinase. In one embodiment, the present invention provides a method of alleviating pain in a subject comprising the step of administering a compound of the present invention to the subject. In other embodiments, the present invention provides a method of alleviating pain in a subject comprising administering the compound of the present invention concomitantly or sequentially with other pharmaceutical agents.

### Definitions

As used herein, the term “subject” refers to an animal, such as a mammal or a human, who has been the object of treatment, observation or experiment.

As used herein, the term “therapeutically effective amount” refers to an amount of crenolanib or pharmaceutically acceptable salt(s) thereof, that elicits the biological or medicinal response in a subject that is being sought by a researcher, veterinarian, medical doctor or other clinician, which includes alleviation of the symptoms of the disease or the disorder being treated, improvement in mobility, or improvement in the function of the subject. Methods for determining therapeutically effective doses for pharmaceutical compositions comprising a compound of the present invention are known in the art.

As used herein, the term “composition” is intended to encompass a product comprising the specified ingredients in the specified amounts, as well as any product which results, directly or indirectly, from combinations of the specified ingredients in the specified amounts.

As used herein, the terms “pain disorder” or “pain” refers to disease or disorders associated with significant pain in a subject, which may limit mobility of function of the subject. Non-limiting examples of such disorders include osteoarthritis pain, chronic pain, neuropathic pain, musculoskeletal pain, acute pain, inflammatory pain, pain associated with a proliferative disease or disorder, idiopathic pain, postsurgical pain, visceral pain, multiple sclerosis, pain associated with bone fracture, trigeminal neuralgia, rheumatoid arthritis, and fibromyalgia.

As used herein, the term “inhibition of TRKA kinase” refers to the activity of the present compound to block phosphorylation and downstream signalling of TRKA kinase, which has been shown to alleviate pain. Further information on TRKA, including full gene and protein sequences, known clinically relevant variants and mutations, tissue expression, and signalling interaction partners can be found at UniProt (accession number P04629-1), GenBank (accession number NM 002529.3, and GenPept (accession number NP 002520.2).

As used herein, the terms “reduces pain” or “eliminates pain” refers to the decrease in objective or subjective measurements of pain in a subject. Methods for determining and scoring levels of pain are known in the art, and include increases in mobility and function, and decreases in allodynia, thermal sensitivity, and self-reported pain on available scaling and/or scoring measures.

In one embodiment, the present invention comprises therapeutically effective amounts of the compound having Formula I:

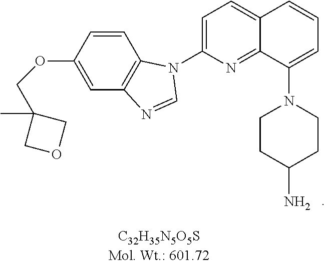 [SMILES: CC1(COc2ccc3c(c2)ncn3-c2ccc3cccc(N4CCC(N)CC4)c3n2)COC1]

or a pharmaceutically acceptable salt or solvate thereof, in a therapeutically effect amount against a pain disorder or pain associated with another disease or disorder. Pain disorders may be selected from at least one of osteoarthritis pain, chronic pain, neuropathic pain, musculoskeletal pain, acute pain, inflammatory pain, pain associated with a proliferative disease or disorder, idiopathic pain, postsurgical pain, visceral pain, multiple sclerosis, pain associated with bone fracture, trigeminal neuralgia, rheumatoid arthritis, and fibromyalgia. Pharmaceutically acceptable salts such as hydrochloride, phosphate and lactate are prepared in a manner similar to the benzenesulfonate salt and are well known to those of moderate skill in the art. The following representative compounds of the present invention are for exemplary purposes only and are in no way meant to limit the invention, including Crenolanib as Crenolanib Besylate, Crenolanib Phosphate, Crenolanib Lactate, Crenolanib Hydrochloride, Crenolanib Citrate, Crenolanib Acetate, Crenolanib Toluenesulphonate and Crenolanib Succinate.

Compounds of the present invention may be administered to a subject systemically, for example, orally, topically, intravenously, subcutaneously, intramuscular, intradermal or parenterally. The compounds of the present invention can also be administered to a subject locally.

Compounds of the present invention may be formulated for slow-release or fast-release with the objective of maintaining contact of compounds of the present invention with targeted tissues for a desired range of time.

Compositions suitable for oral administration include solid forms, such as pills, tablets, caplets, capsules, granules, and powders, liquid forms, such as solutions, emulsions, and suspensions. Forms useful for parenteral administration include sterile solutions, emulsions and suspensions.

The daily dosage of the compounds of the present invention may be varied over a wide range from 50 to 500 mg per adult human per day. For oral administration, the compositions are preferably provided in the form of tablets containing 20 and 100 milligrams. The compounds of the present invention may be administered on a regimen up to three times or more per day. Preferably three times per day. Optimal doses to be administered may be determined by those skilled in the art, and will vary with the compound of the present invention used, the mode of administration, the time of administration, the strength of the preparation, the details of the disease condition. Factors associated with patient characteristics, such as age, weight, and diet will call for dosage adjustments. In other examples, the daily dosage of the compounds of the present invention may be varied over a wide range from 15 to 500, 25 to 450, 50 to 400, 100 to 350, 150 to 300, 200 to 250, 15, 25, 50, 75, 100, 150, 200, 250, 300, 400, 450, or 500 mg per day. The compounds of the present invention may be administered on a daily regimen, once, twice, three or more times per day. Optimal doses to be administered may be determined by those skilled in the art, and will vary with the compound of the present invention used, the mode of administration, the time of administration, the strength of the preparation, the details of the disease condition. One or more factors associated with subject characteristics, such as age, weight, and diet will call for dosage adjustments. Techniques and compositions for making useful dosage forms using the Crenolanib are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); relevant portions incorporated herein by reference.

A dosage unit for use of Crenolanib, may be a single compound or mixtures thereof with other compounds, e.g., a potentiator. The compounds may be mixed together, form ionic or even covalent bonds. The compounds of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the Crenolanib of the present invention to a patient in need of therapy.

The Crenolanib is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the Crenolanib may be formulated to provide, e.g., maximum and/or consistent dosing for the particular form for oral, rectal, topical, intravenous injection, subcutaneous injection, intramuscular injection, or parenteral administration. While the Crenolanib may be administered alone, it will generally be provided in a stable salt form mixed with a pharmaceutically acceptable carrier. The carrier may be solid or liquid, depending on the type and/or location of administration selected.

Preparation of the compounds of the present invention. General synthetic methods, which may be referred to for preparing the compounds of formula I are provided in U.S. Pat. No. 5,990,146 (issued Nov. 23, 1999) (Warner-Lambert Co.) and PCT published application numbers WO 99/16755 (published Apr. 8, 1999) (Merck & Co.) WO 01/40217 (published Jul. 7, 2001) (Pfizer, Inc.), US Patent Application No. US 2005/0124599 (Pfizer, Inc.) and U.S. Pat. No. 7,183,414 (Pfizer, Inc.), relevant portions incorporated herein by reference.

Pharmaceutically acceptable salts such as hydrochloride, phosphate and lactate are prepared in a manner similar to the benzenesulfonate salt and are well known to those of moderate skill in the art. The following representative compounds of the present invention are for exemplary purposes only and are in no way meant to limit the invention.

Non-limiting examples of other pharmaceutical agents which may be administered concomitant or sequentially with crenolanib include opioid analgesics, nonsteroidal anti-inflammatory drugs, barbiturate sedatives, benzodiazepines having sedative action, histamine antagonists having sedative action, sedatives, skeletal muscle relaxants, tricyclic antidepressants, anticonvulsants, COX-2 selective inhibitors, coal-tar analgesics, neuroleptics, beta-adrenergics, local anesthetics, corticosteroids, 5-HT receptor agonists or antagonists, cholinergic analgesics, tramadol, PDE5 inhibitors, alpha-2-delta ligands, cannabinoids, metabotropic glutamate subtype 1 receptor antagonists, serotonin reuptake inhibitors, noradrenaline reuptake inhibitors, dual serotonin-noradrenaline reuptake inhibitors, inducible nitric oxide synthase inhibitors, acetylcholinesterase inhibitors, 5-lipoxygenase inhibitors, sodium channel blockers, Naᵥ1.7 blockers, Naᵥ1.8 blockers, 5-HT3 antagonists, TPRV1 receptor agonists, nicotinic receptor antagonists, nerve growth factor antagonists, endopeptidase stimulants, and angiotensin II antagonists.

Biological Activity

In vitro Assays. The following representative in vitro assays were performed in determining the TRKA/TRKB/TRKC biological activity of the present invention. These are given to illustrate the invention in a non-limiting fashion.

Inhibition of TRK enzyme activity exemplifies the specific inhibition of the TRK enzyme and cellular processes that are dependent on TRK activity. All of the examples herein show significant and specific inhibition of TRK kinase and TRK-dependent cellular responses.

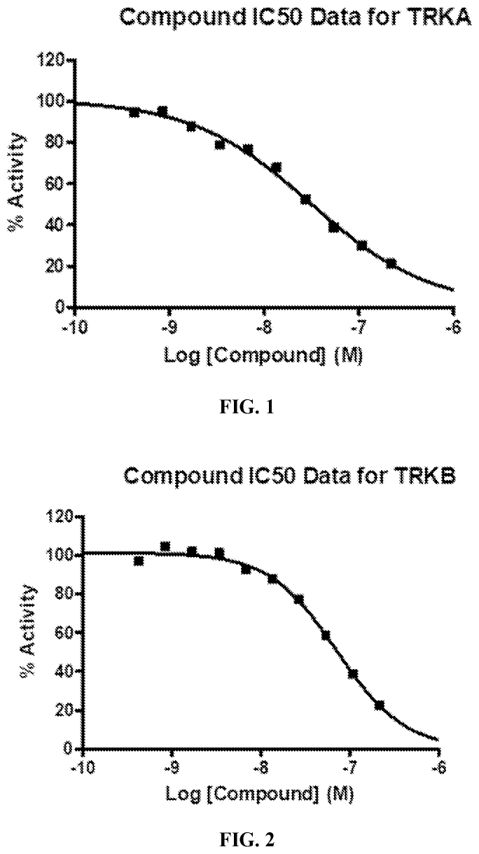

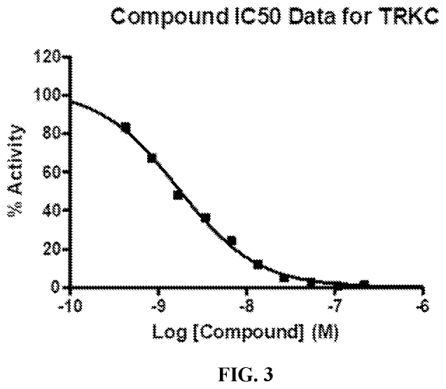

Direct enzyme phosphorylation assay. The Reaction Biology HotSpot Kinase assay was used to screen the present invention against a panel of TRKA, TRKB, and TRKC kinases. For assays of all kinases, the TRK enzyme was prepared in base reaction buffer (20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO). The reaction was initiated by the addition of 33P-ATP (10 μCi/μL) into the mixture. The reaction mixture was incubated for 120 minutes at room temperature. Radioactivity was detected by filter-binding method, and kinase activity expressed as the percent remaining kinase activity in test samples compared to vehicle reactions. IC50 values and curve fitting were obtained using Prism (GraphPad Software). The activity of the besylate salt of the present invention was determined using a direct enzymatic Reaction Biology HotSpot Kinase assay. All IC50 values are presented in nanomolar concentration. In the direct enzymatic measurement assay, the IC50 of the besylate salt of the current invention against the various TRK kinases is shown in Table 1. The activity of the besylate salt of the present invention against these kinases is also displayed in FIGS. 1 to 13.

TABLE 1

| Kinase | IC50 (nM) |
|---|---|
| TRKA | 31.0 |
| TRKB | 77.7 |
| TRKC | 1.68 |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±0.1, 0.5, 1, 2, 3, 4, 5, 6, 7, 10, 12 or 15%, or as understood to be within a normal tolerance in the art, for example, within 2 standard deviations of the mean. Unless otherwise clear from the context, all numerical values provided herein are modified by the term about.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe 30 the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Aikawa, T., N. Togashi, K. Iwanaga, H. Okada, Y. Nishiya, S. Inoue, M. J. Levis and T. Isoyama (2020). “Quizartinib, a selective FLT3 inhibitor, maintains antileukemic activity in preclinical models of RAS-mediated midostaurin-resistant acute myeloid leukemia cells.” Oncotarget 11(11).
Aloe, L., M. A. Tuveri, U. Carcassi and R. Levi-Montalcini (1992). “Nerve growth factor in the synovial fluid of patients with chronic arthritis.” Arthritis Rheum 35(3): 351-355.
Amatu, A., A. Sartore-Bianchi, K. Bencardino, E. G. Pizzutilo, F. Tosi and S. Siena (2019). “Tropomyosin receptor kinase (TRK) biology and the role of NTRK gene fusions in cancer.” Annals of Oncology 30: viii5-viii15.
Ashraf, S., K. S. Bouhana, J. Pheneger, S. W. Andrews and D. A. Walsh (2016). “Selective inhibition of tropomyosin-receptor-kinase A (TrkA) reduces pain and joint damage in two rat models of inflammatory arthritis.” Arthritis Res Ther 18(1): 97.
Dakin, P., S. J. DiMartino, H. Gao, J. Maloney, A. J. Kivitz, T. J. Schnitzer, N. Stahl, G. D. Yancopoulos and G. P. Geba (2019). “The Efficacy, Tolerability, and Joint Safety of Fasinumab in Osteoarthritis Pain: A Phase IIb/III Double-Blind, Placebo-Controlled, Randomized Clinical Trial.” Arthritis Rheumatol 71(11): 1824-1834.
Einarsdottir, E., A. Carlsson, J. Minde, G. Toolanen, O. Svensson, G. Solders, G. Holmgren, D. Holmberg and M. Holmberg (2004). “A mutation in the nerve growth factor beta gene (NGFB) causes loss of pain perception.” Hum Mol Genet 13(8): 799-805.
Goldberg, A. (2020). Younger Patients with Newly Diagnosed FLT3-Mutant AML Treated with Crenolanib Plus Chemotherapy Achieve Durable Remissions. EHA.
Hansel, T. T., H. Kropshofer, T. Singer, J. A. Mitchell and A. J. George (2010). “The safety and side effects of monoclonal antibodies.” Nat Rev Drug Discov 9(4): 325-338.
Hirose, M., Y. Kuroda and E. Murata (2016). “NGF/TrkA Signaling as a Therapeutic Target for Pain.” Pain Pract 16(2): 175-182.
Hochberg, M. C., L. A. Tive, S. B. Abramson, E. Vignon, K. M. Verburg, C. R. West, M. D. Smith and D. S. Hungerford (2016). “When Is Osteonecrosis Not Osteonecrosis?: Adjudication of Reported Serious Adverse Joint Events in the Tanezumab Clinical Development Program.” Arthritis Rheumatol 68(2): 382-391.
Indo, Y., M. Tsuruta, Y. Hayashida, M. A. Karim, K. Ohta, T. Kawano, H. Mitsubuchi, H. Tonoki, Y. Awaya and I. Matsuda (1996). “Mutations in the TRKA/NGF receptor gene in patients with congenital insensitivity to pain with anhidrosis.” Nat Genet 13(4): 485-488.
Lewis, N. L., L. D. Lewis, J. P. Eder, N. J. Reddy, F. Guo, K. J. Pierce, A. J. Olszanski and R. B. Cohen (2009). “Phase I study of the safety, tolerability, and pharmacokinetics of oral CP-868,596, a highly specific platelet-derived growth factor receptor tyrosine kinase inhibitor in patients with advanced cancers.” J Clin Oncol 27(31): 5262-5269.
Loeser, J. D. and R. D. Treede (2008). “The Kyoto protocol of IASP Basic Pain Terminology.” Pain 137(3): 473-477.
Mantyh, P. W., M. Koltzenburg, L. M. Mendell, L. Tive and D. L. Shelton (2011). “Antagonism of nerve growth factor-TrkA signaling and the relief of pain.” Anesthesiology 115(1): 189-204.
Petty, B. G., D. R. Cornblath, B. T. Adornato, V. Chaudhry, C. Flexner, M. Wachsman, D. Sinicropi, L. E. Burton and S. J. Peroutka (1994). “The effect of systemically administered recombinant human nerve growth factor in healthy human subjects.” Ann Neurol 36(2): 244-246.
Sarchielli, P., M. L. Mancini, A. Floridi, F. Coppola, C. Rossi, K. Nardi, M. Acciarresi, L. A. Pini and P. Calabresi (2007). “Increased levels of neurotrophins are not specific for chronic migraine: evidence from primary fibromyalgia syndrome.” J Pain 8(9): 737-745.
Schnitzer, T. J., R. Easton, S. Pang, D. J. Levinson, G. Pixton, L. Viktrup, I. Davignon, M. T. Brown, C. R. West and K. M. Verburg (2019). “Effect of Tanezumab on Joint Pain, Physical Function, and Patient Global Assessment of Osteoarthritis Among Patients With Osteoarthritis of the Hip or Knee: A Randomized Clinical Trial.” JAMA 322(1): 37-48.
von Mehren, M., E. D. Tetzlaff, M. Macaraeg, J. Davis, A. Vartika, R. Abhijit and M. C. Heinrich (2016). “Dose escalating study of crenolanib besylate in advanced GIST patients with PDGFRA D842V activating mutations.” J Clin Oncol 34 (suppl; abstr 11010).

What is claimed is:

1. A method for reducing or eliminating pain in a subject comprising administering to the subject a therapeutically effective amount of crenolanib or a pharmaceutically acceptable salt thereof wherein the crenolanib or salt thereof to reduce or eliminate pain.

2. The method of claim 1, wherein the inhibition of TRKA kinase attenuates or ameliorates pain.

3. The method of claim 1, wherein the pain is selected from at least one of osteoarthritis pain, chronic pain, neuropathic pain, musculoskeletal pain, acute pain, inflammatory pain, pain associated with a proliferative disease or disorder, idiopathic pain, postsurgical pain, visceral pain, multiple sclerosis, pain associated with bone fracture, trigeminal neuralgia, rheumatoid arthritis, migraine pain, or fibromyalgia.

4. The method of claim 1, wherein the therapeutically effective amount or crenolanib or the pharmaceutically acceptable salt thereof is from about 50 to 800 mg per day, 100 to 450 mg per day, 200 to 400 mg per day, 300 to 500 mg per day, 350 to 500 mg per day, 400 to 600 mg per day, or 600 to 800 mg per day.

5. The method of claim 1, wherein the therapeutically effective amount of crenolanib or the pharmaceutically acceptable salt thereof is administered at least one of continuously, intermittently, systemically, or locally.

6. The method of claim 1, wherein the crenolanib or the pharmaceutically acceptable salt thereof is administered orally, intravenously, intraperitoneally, intramuscularly, subcutaneously, or topically.

7. The method of claim 1, wherein the crenolanib or the pharmaceutically acceptable salt thereof is administered as a single agent, or concomitantly or sequentially with another pharmaceutical agent.

8. The method of claim 1, wherein the crenolanib is a pharmaceutically acceptable salt of crenolanib.

9. The method of claim 1, wherein the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, crenolanib tosylate, crenolanib mesylate, crenolanib benzoate, or crenolanib succinate.

10. The method of claim 1, wherein the therapeutically effective amount of crenolanib or the pharmaceutically acceptable salt thereof is administered up to three times or more a day for as long as the subject is in need of treatment.

11. A method for reducing or eliminating pain in a subject comprising identifying a subject in need of reducing or eliminations pain and administering to the subject a therapeutically effective amount of crenolanib or a pharmaceutically acceptable salt thereof wherein the crenolanib or salt thereof to reduce or eliminate pain.

12. The method of claim 11, wherein the inhibition of TRKA kinase attenuates or ameliorates pain.

13. The method of claim 11, wherein the pain is selected from at least one of osteoarthritis pain, chronic pain, neuropathic pain, musculoskeletal pain, acute pain, inflammatory pain, pain associated with a proliferative disease or disorder, idiopathic pain, postsurgical pain, visceral pain, multiple sclerosis, pain associated with bone fracture, trigeminal neuralgia, rheumatoid arthritis, migraine pain, or fibromyalgia.

14. The method of claim 11, wherein the therapeutically effective amount or crenolanib or the pharmaceutically acceptable salt thereof is from about 50 to 800 mg per day, 100 to 450 mg per day, 200 to 400 mg per day, 300 to 500 mg per day, 350 to 500 mg per day, 400 to 600 mg per day, or 600 to 800 mg per day.

15. The method of claim 11, wherein the therapeutically effective amount of crenolanib or the pharmaceutically acceptable salt thereof is administered at least one of continuously, intermittently, systemically, or locally.

16. The method of claim 11, wherein the crenolanib or the pharmaceutically acceptable salt thereof is administered orally, intravenously, intraperitoneally, intramuscularly, subcutaneously, or topically.

17. The method of claim 11, wherein the crenolanib or the pharmaceutically acceptable salt thereof is administered as a single agent, or concomitantly or sequentially with another pharmaceutical agent.

18. The method of claim 11, wherein the crenolanib is a pharmaceutically acceptable salt of crenolanib.

19. The method of claim 11, wherein the crenolanib or the pharmaceutically acceptable salt thereof is crenolanib besylate, crenolanib phosphate, crenolanib lactate, crenolanib hydrochloride, crenolanib citrate, crenolanib acetate, crenolanib toluenesulphonate, crenolanib tosylate, crenolanib mesylate, crenolanib benzoate, or crenolanib succinate.

20. The method of claim 11, wherein the therapeutically effective amount of crenolanib or the pharmaceutically acceptable salt thereof is administered up to three times or more a day for as long as the subject is in need of treatment.
